(19) United States
(12) Patent Application Publication
(10) Pub. No.: US 2021/0390480 A1
(43) Pub. Date: Dec. 16, 2021

(54) METHOD AND DEVICE FOR PRODUCTION SCHEDULING OF NUTRITIONAL TABLET, ELECTRONIC EQUIPMENT AND STORAGE MEDIUM

(71) Applicant: IVC Nutrition Corporation, Taizhou (CN)

(72) Inventors: Xiaoyun XU, Taizhou (CN); Kewei JIANG, Taizhou (CN); Haidong LI, Taizhou (CN); Bokai TIAN, Taizhou (CN); Xudong XIA, Taizhou (CN); Chendong SHANG, Taizhou (CN); Yaping ZHAO, Taizhou (CN); Zhen LI, Taizhou (CN); Yan WANG, Taizhou (CN)

(21) Appl. No.: 17/459,109

(22) Filed: Aug. 27, 2021

Related U.S. Application Data
(63) Continuation of application No. PCT/CN2021/078637, filed on Mar. 2, 2021.

(30) Foreign Application Priority Data
May 19, 2020 (CN) 202010422666.1

(51) Int. Cl. G06Q 10/06; G16H 40/20; G06Q 50/04; G06Q 10/08; G06F 30/20

(52) U.S. Cl. CPC G06Q 10/06313; G16H 40/20; G06Q 50/04; G06F 2111/10; G06Q 10/063118; G06Q 10/067; G06F 30/20; G06Q 10/0875

(57) ABSTRACT

Provided are a method and a device for production scheduling of nutritional tablets, an electronic equipment and a storage medium. In the method, pending order information and production information of the nutritional tablets, and constraint conditions jointly formed thereby are acquired. A production scheduling model is constructed according to the constraint conditions, and then the pending order information and the production information are input into the production scheduling model to obtain a production scheduling scheme.

### CROSS-REFERENCE TO RELATED APPLICATIONS

This application is a continuation of International Patent Application No. PCT/CN2021/078637, filed on Mar. 2, 2021, which claims the benefit of priority from Chinese Patent Application No. 202010422666.1, filed on May 19, 2020. The content of the aforementioned applications, including any intervening amendments thereto, is incorporated herein by reference.

### TECHNICAL FIELD

This application relates to nutrient production, and more particularly to a method and a device for production scheduling of nutritional tablets, electronic equipment and storage mediums.

### BACKGROUND

The production scheduling of nutritional tablets generally involves a huge number of products, molds and machines with different productivities in the production site. In view of this, the linkage of multiple production links together with inventory constraints and a large number of regulatory requirements is needed to be considered in the production process. Accordingly, the manual production scheduling method will easily be affected by the complex production conditions, which will result in poor cooperative optimization of various production links, causing order delays.

### SUMMARY

An object of this application is to provide methods and devices for production scheduling of nutritional tablets, electronic equipment and storage mediums to solve the problems in the prior art that the manual production scheduling method is susceptible to the complex production conditions, resulting in poor cooperative optimization of various production links and order delay.

In the first aspect, this application provides a production scheduling method of nutritional tablets, comprising:

acquiring pending order information and production information of the nutritional tablets, and constraint conditions jointly formed by the pending order information and the production information;

constructing a production scheduling model according to the constraint conditions; and

inputting the pending order information and the production information into the production scheduling model to obtain a production scheduling scheme.

In the second aspect, this application provides a device for production scheduling of nutritional tablets, comprising:

an acquiring module;

a building module; and

an outputting module;

wherein the acquiring module is configured to obtain pending order information, production information of the nutritional tablets, and constraint conditions jointly formed by the pending order information and the production information;

the building module is configured to build a production scheduling model according to the constraint conditions; and

the outputting module is configured to input the pending order information and the production information into the production scheduling model to output a production scheduling scheme.

In the third aspect, this application provides an electronic device, comprising:

a processor; and

a memory;

wherein the memory stores a program code; and the processor is configured to execute the above production scheduling method through executing the program code.

In the fourth aspect, this application provides a computer-readable storage medium, comprising:

a program code;

wherein the program code is configured to drive an electronic device to execute the production scheduling method when being run on the electronic device.

Compared to the prior art, this application has the following beneficial effects.

In this application, a production scheduling model is constructed according to the constraint conditions formed by the pending order information and production information, so that various production steps are linked, thereby improving the collaborative optimization of various production steps and effectively avoiding the order delay.

### BRIEF DESCRIPTION OF THE DRAWINGS

The disclosure will be further described below in detail with reference to the accompanying drawings and the embodiments.

FIG. 1 is a flow chart of the production scheduling method of nutritional tablets according to the embodiment of the disclosure.

FIG. 2 is a main flow chart of nutritional tablet production according to the embodiment of the disclosure.

FIG. 3 schematically depicts the construction of the production scheduling model according to the embodiment of the disclosure.

FIG. 4 schematically illustrates comparisons between manual production scheduling and model production scheduling according to the embodiment of the disclosure.

FIG. 5 is a structural block diagram of devices for production scheduling of nutritional tablets according to the embodiment of the disclosure.

### DETAILED DESCRIPTION OF EMBODIMENTS

The disclosure will be described in detail with reference to the accompanying drawings. These drawings are all simplified schematic diagrams, which are merely illustrative of the basic structure of the disclosure and only show the structures related to the disclosure.

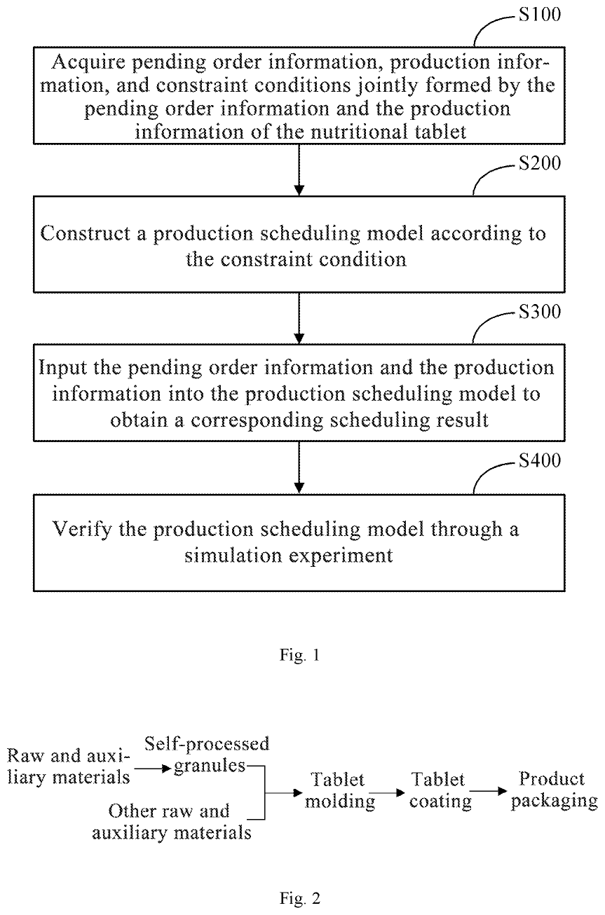

As shown in FIG. 1, this application provides a production scheduling method of nutritional tablets, which has the following specific steps.

S100 Pending order information and production information of the nutritional tablets, and constraint conditions jointly formed by the pending order information and the production information are acquired.

In some embodiments, the pending order information includes order time, product name, product quantity, order priority and delivery date.

In some embodiments, the production information includes machine list information, machine scheduling information, mold information, processing speed information of each machine, cleaning rule information, customer priority information, workshop personnel scheduling information, product information, auxiliary equipment information, follow-up inspection and logistics time information, output target and scheduling cycle information, product batch information, raw material inventory and arrival information.

In some embodiments, machine types include tablet press, granulator and pill. Machine scheduling information includes mechanical start time, shifts and refurbishment. Mold information includes a mold list corresponding to each product and a mold list applicable for each machine. Cleaning rule information includes different products, machines, allergens, and large and small cleaning rules of the mold. Product information includes shape, color, packaging and corresponding mold requirements of the products.

The operation process of production of nutritional tablet is sorted out.

Based on the analysis of the production site and related data, the operation process of nutritional tablet production is sorted out first. As shown in FIG. 2, the production process of tablets is mainly a physical process, and the production process involves self-processed granules and raw materials purchased from other suppliers. The granules can be self-processed or purchased, and the types of self-processed granules will be controlled based on factors such as cost and management complexity. In addition to granules, other raw and auxiliary materials need to be added, such as low-content functional ingredients, some excipients in the tableting process, flavors and fragrances, colorings, etc. After all the raw materials and auxiliary materials, granules and other intermediates are prepared, the fully formulated materials are mixed according to the process to ensure uniformity. The production of nutritional tablets is carried out in batches. After the tableting is finished, the surface of some tablet products need to be coated with a film due to stability problems or customer requirements. After the production of the nutritional tablet is finished, the final packaging is carried out according to the sales channel.

The production scheduling situation of nutritional tablets is analyzed.

The business side provides product requirements to the production planning department. The requirements are in various forms, and mainly include the following two modes. (1) The product name, product quantity and delivery date are provided, and the colleagues in the planning department will directly schedule production accordingly. If the delivery date is indeed not met, the feedback is conveyed to the corresponding business department to confirm whether to postpone or cancel the order. (2) The customer information, product name and product quantity (mainly for external customers) are provided, and the planning department will schedule the expected delivery date according to customer priority and capacity. The expected delivery date is given to the business department as the delivery date promised to the customer.

Each product has a corresponding formula bill of materials (BOM), based on which the number of granular intermediates and the number of raw and auxiliary materials required can be decomposed. Granules and raw and auxiliary materials have corresponding inspection and release time, and the scheduling of the tableting must be after the release of intermediates and raw materials. On-site production scheduling is done by listing the production capacity of all tablet presses, adhering to the principle of “first come, first served” orders, and complete the “rough scheduling” of orders. The so-called “rough scheduling” means that the production planner lists the types of products and the number of batches that need to be completed every day for each production line in each workshop according to the order delivery status. After the rough scheduling is applied to the workshop production site, the on-site management personnel will also determine the order of production for the products on a production line according to the actual situation on the site, and form the final executable “fine scheduling” in the production workshop. The rough scheduling cycle is generally fixed, but sometimes the orders will be adjusted, especially temporary orders insertion and adjustment for customers with high customer priority. Therefore, the rough scheduling of the production department is actually carried out every day.

The production and delivery of nutritional tablets are diagnosed.

The overall delivery level of nutritional tablets is average, and the main reasons are as follows. (1) Capacity factor: the production capacity of nutritional tablet is usually very tight, and the production scheduling method of manual scheduling is far from the “optimal solution”. (2) Raw material factors: 10-30 kinds of raw and auxiliary materials are used in the formula of each nutritional tablet product, and different products may use a certain raw and auxiliary materials. Therefore, raw material not arriving on time will affect the normal production of multiple products. At the same time, the transparency of the nutrition industry is getting higher and higher. The customer will specify the supplier of more and more raw materials, and some even directly provide raw materials. This situation is not only a great challenge to the purchasing side, but also a great reduction of flexibility in production arrangement. (3) Logistics factors: various delivery forms and a wide range of business areas have caused greater challenges to the logistics of nutritional tablet products. It is very likely that the goods will be backlogged in the port and the customs clearance time will be long. In order to deliver on time, it causes more pressure on the processing and delivery time of production links. Regarding the above-mentioned influencing factors, the capacity factor accounts for an absolutely high proportion and has a huge impact, it is also a long-term bottleneck of production and operation. Therefore, this application aims at optimizing the capacity by studying the production scheduling model, improving the order delivery level.

S200 The production scheduling model is constructed according to the constraint conditions.

For the optimization of the scheduling of nutritional tablets, the goal of this embodiment is to satisfy all constraint conditions according to the customer demand for nutritional tablet products, optimize the product scheduling, design a fully automatic scheduling model for the production of nutritional tablet products, and output scheduling information that can be used by the production workshop when minimizing the number of days the order is cumulatively delayed.

Considering the actual production requirements and the operability of the scheduling model, the following constraints should be met. (1) Material completeness: the raw materials required to manufacture the granules are all prepared before the production of the granules. Before the production of the nutritional tablet products, the amount of granules and other raw materials required to produce the product are prepared and certified. (2) Clearance rules: site clearance refers to job operators cleaning and tidying up the production environment, equipment, containers, utensils, documents, etc., to ensure that there are no remnants from the previous production and to achieve the clean state before production. The production of nutritional tablet products requires large-scale clearance when switching product types or continuous production of the same product exceeding several batches or quantities. The time of clearance is related to the machine model, whether it involves changing the mold, whether the color of the nutritional tablet product changes from dark to light, etc. The production of granules requires a large-scale clearance when switching granule types. The time of clearance is related to the properties of the granules such as whether it is calcium or colored. (3) Correspondence between nutritional tablet and granules: granules are used as intermediates in the production of nutritional tablet. One nutritional tablet may use a plurality of granules as intermediates, and one granule may also be used to produce multiple types of nutritional tablet. The granules are prepared before the nutritional tablet, and the corresponding nutritional tablet cannot enter the production when the completeness of the granules is not satisfied. (4) Work calendar: no production will be arranged during the holiday. (5) Correspondence between products and equipment: according to product characteristics and equipment performance, there is a matching relationship between granules and granulators and between nutritional tablet and tablet presses. That is, a certain product and a certain granule can be processed and produced only on a certain machine. For example, some granules requiring alcohol granulation must be produced on explosion-proof granulation lines. (6) Production restriction of granulating line; the same type of granulating line needs to produce the same product at the same time (because the same types of the granulating line are arranged in one room, it is necessary to avoid cross-contamination between products). (7) Correspondence between nutritional tablets, molds, and tablet presses: a certain product needs a specific mold on a certain machine. The shape requirements of customers for nutritional tablet products determine the choice of molds. A corresponding relationship is existed between the molds and tablet press. That is, even if the same product is produced on different models of tablet presses, the molds are different. (8) Limit of the number of molds: the sum of the number of a mold used on all machines on the same day cannot exceed the number of the mold. (9) Inspection and release: the delivery time of the product order should be minus 7 days of inspection and release time, that is, the production of nutritional tablet should be completed 7 days before the delivery time.

The variables of the nutritional tablet scheduling model mainly cover the types of materials (raw materials, intermediates and finished products), equipment types (quantity and corresponding clearance time), product correspondence (tablets and granules), clearance rules, customer priority, production efficiency, etc. The above variable information can be adjusted in real time in the production scheduling model. If the number of equipment is increased or decreased, and the production efficiency is improved, the model can be updated immediately. Four assumptions are proposed according to the characteristics of nutritional tablet and granule production. (1) The tablet pressing process of the nutritional tablets ends with a clearance every day. Actually, the production line is cleared in accordance with the clearance rules stipulated by the Quality Department, and it is not necessary to conduct a daily clearance. The main purpose of defining this hypothetical condition is that the actual production site often has various emergencies, such as shortage of materials, abnormal equipment requiring maintenance, etc., so the daily clearance time is added to the production scheduling model to reserve time for handling of these emergencies. In addition, when more than one product is produced every day, the production sequence is also involved. In cross-day production, the production sequence may have a significant impact on the large clearance time. However, the introduction of a fine production sequence will make the volume of the entire scheduling model 2-3 times the original volume, which will significantly affect the solution speed for industrial-level applications. When the hypothesis of daily clearance time is added, a natural isolation of the production between adjacent days is formed, which can greatly reduce the complexity of the model. (2) The remaining time of the schedule before 24:00 on the same day is included in the next day. The workshop arranges three shifts in 24 h every day, each shift is 8 h. After a machine completes all the orders scheduled on the day, the remaining time can be counted into the next day. As long as the raw materials for the subsequent orders are all set and the molds are available, the subsequent orders can continue to be produced. Making this assumption is more in line with the actual situation on site. (3) The raw materials arriving before 24:00 on the same day need to be stored in the warehouse and included in the raw material inventory at the beginning of the next day. All raw materials must be stored in the warehouse and released before use. Therefore, it is assumed that all raw materials arriving on the same day are included in the inventory data of next day, which is in line with the actual production situation. (4) The granules produced before 24:00 on the same day need to be stored in the warehouse and included in the granules inventory at the beginning of the next day. As same as the raw material inventory data assumption, the amount of self-processed granules stored in the warehouse on the same day will be included in the granule inventory data of the next day.

Based on the above analysis and setting of objectives, constraints, variables and assumptions, the following scheduling model for the production process of nutritional tablet products are established.

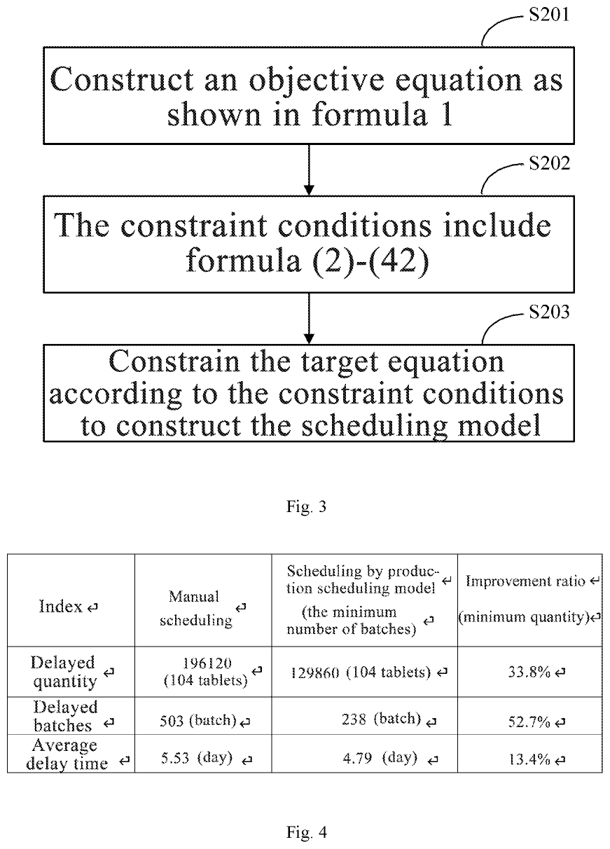

As shown in FIG. 3, S200 has the following steps.

S201 An objective function is constructed, as shown in formula (1), which represents the minimization of the cumulative weighted delayed delivery order quantity.

min Σⱼ₌₁^{J}Σₜ₌₁^{T}(pⱼ×vⱼₜ); (1);

where j is a serial number of products, and is an integer selected from 1-J; t is a time number with a unit of day, and production is arranged from the first day; t is an integer selected from 1-T; T is order production scheduling cycle; pⱼ is production priority of product j; and Uⱼₜ is delay amount of the product j on the t^{th} day.

The constraint conditions comprise formulas (2)-(42) shown as follows.

Formula (2) represents a daily capacity constraint of granulator on weekdays;

s.t. Σₖ₌₁^{K}(xgₖₘₜ×PGTₖₘ+xgaₖₘₜ×SATGₘ)+sgₘₜ=24,∀m=1, . . . ,M,t=1, . . . ,T,WDₜ=1; (2);

where s is a serial number of mold types, and s is an integer selected from 1-S; k is a serial number of granules, and k is an integer selected from 1-K; xgₖₘₜ is the number of batches of granules k processed on a granulator m on the t^{th} day; PGTₖₘ is one pot processing time of the granules k; processing time of granules that cannot be processed by the granulator m is regarded as 0; SATGₘ is time for single clearing on the granulator m during switching of granules; sgₘₜ is idle time of the granulator m at the t^{th} day; m is a serial number of a granulator, and m is an integer selected from 1-M; and WDₜ is a date for rest, and t is an integer selected from 1-T.

Formulas (3)-(6) represent the daily capacity constraints of the tablet press on weekdays;

Σ_{j=1,MP}_{ⱼ}_{≤150}^{J}(xtⱼₙₜ×(MPⱼ/PTⱼₙ+SITₙ/SCₙ)+xtaⱼₙₜ×SATTₙ−xtaⱼₙₜ×SITₙ/SCₙ)+Σ_{j=1,MP}_{ⱼ}_{>150}^{J}(xtⱼₙₜ×(MPⱼ/PTⱼₙ+SITₙ)+xtaⱼₙₜ×SATTₙ−xtaⱼₙₜ×SITₙ)+stₙₜ=24+Coprₙₜ×CLTₙ+(1−savₙₜ)×SAVTTNₙ+(1−sav2ₙₜ)×SAVTTNₙ;n=1, . . . 5,t=1 . . . ,T−1,WDₜ=1 (3);

where MPⱼ is a single-batch output of product j; for two machines manufacturing the same product in a workshop, output of the two machines is calculated as double an one-pot output of one of the two machines; xtⱼₙₜ is the number of batches of product j manufactured on tablet press n on the t^{th} day; PTⱼₙ is the processing speed of product j on tablet press n; processing speed of a product that fails to be processed by the tablet press n is regarded as 0; SITₙ is time for a single small cleaning for the tablet press n; SCₙ is a frequency of small cleaning for tablet press n, which is calculated by the number of batches of the same product that have been produced when one small cleaning is required; xtaⱼₙₜ is 1 if product j is produced on tablet press n on the t^{th} day, otherwise xtaⱼₙₜ is 0; SATTₙ is the longest time used for a single large clearing during product switching on tablet press n; stₙₜ is the idle time of tablet press n on the t^{th} day; Coprₙₜ is 1 if the color of a product produced on the same machine on two consecutive days is changed from dark to light, otherwise Coprₙₜ is 0; CLTₙ is the time required for switching color of a product on tablet press n; savₙₜ is 1 if the same mold is used on the same machine for two consecutive days, otherwise savₙₜ is 0; SAVTTₙ is the time required for mold switching on the tablet press n, and is also the time for large clearing; and n is a serial number of the tablet press, and is an integer selected from 1-N.

$$\begin{matrix}{\sum }_{j = 1 , {MP}_{j} \leq 150}^{J} ( {xt}_{jnt} \times ( {MP}_{j} \text{/} {PT}_{jn} + {SIT}_{n} \text{/} {SC}_{n} ) + {xta}_{jnt} \times {SATT}_{n} - {xta}_{jnt} \times {SIT}_{n} \text{/} {SC}_{n} ) + {\sum }_{j = 1 , {MP}_{j} > 150}^{J} ( {t}_{jnt} \times ( {MP}_{j} \text{/} {PT}_{jn} + {SIT}_{n} ) + {xta}_{jnt} \times {SATT}_{n} - {xta}_{jnt} \times {SIT}_{n} ) + {st}_{nt} = 24 + {Copr}_{nt} \times {CLT}_{n} + ( 1 - {sav}_{nt} ) \times {SAVTTN}_{n} + ( 1 - sav {2}_{nt} ) \times {SAVTTN}_{n} ; n = 6 , … , 9 , t = 1 , … , T - 1 , {WD}_{t} = 1 ; ; & ( 5 ) \\ {\sum }_{j = 1 , {MP}_{j} \leq 150}^{J} ( {xt}_{jnt} \times ( {MP}_{j} \text{/} {PT}_{jn} + {SIT}_{n} \text{/} {SC}_{n} ) + {xta}_{jnt} \times {SATT}_{n} - {xta}_{jnt} \times {SIT}_{n} \text{/} {SC}_{n} ) + {\sum }_{j = 1 , {MP}_{j} > 150}^{J} ( {t}_{jnt} \times ( {MP}_{j} \text{/} {PT}_{jn} + {SIT}_{n} ) + {xta}_{jnt} \times {SATT}_{n} - {xta}_{jnt} \times {SIT}_{n} ) + {st}_{nt} = 24 n = 6 , … , 9 , t = T , {WD}_{t} = 1 ; . & ( 6 )\end{matrix}$$

Formula (7) represents the release constraint of raw materials, and the product will not be produced before the release date of raw materials;

Σⱼ₌₁^{J}Σₙ₌₁^{N}xtaⱼₙₜ=0,∀t=1, . . . ,T,WDₜ=0; (7)

Formula (8) represents the rest constraint, and no production is carried out on the rest day;

Σₜ₌₁^{FX}^{ⱼ}⁻¹Σₙ₌₁^{N}xtaⱼₙₜ=0,∀j=1, . . . ,J; (8),

where FXⱼ is a release date of raw materials, and j is an integer selected from 1-J.

Formulas (9)-(10) represent that the inventory of product j at the beginning of t^{th} day is equal to the inventory at the beginning of the previous day plus the production of the previous day minus the demand of the previous day;

IFⱼ₁=0,∀j=1, . . . ,J; (9);

where IFjt is the inventory of product j at the beginning of the t^{th} day;

IFⱼₜ=IFⱼ₍ₜ₋₁₎+Σₙ₌₁^{N}(xtⱼₙ₍ₜ₋₁₎×MPⱼ)−DMTⱼ₍ₜ₋₁₎,∀j=1, . . . ,J,t=2, . . . ,T+1; (10);

where DMTⱼₜ is the demand of product j at the t^{th} day.

Formulas (11)-(12) represent that when the inventory of product j is negative on t^{th} day, the delay is its opposite; when the inventory is non negative, the delay is 0;

uⱼₜ≥−IFⱼ₍ₜ₊₁₎,∀j=1, . . . ,J,t=1, . . . ,T; (11);

uⱼₜ≥0,∀j=1, . . . ,I,t=1, . . . ,T; (12).

Formula 13 represents that when the delay occurs on t^{th} day for product j, it is considered as an order delay;

uⱼₜ/MPⱼ≤vⱼₜ×M₃,∀j=1, . . . ,J,t=1, . . . ,T; (13);

where M₁₋₃ is a constant.

Formulas (14)-(15) represent the raw material inventory at the beginning of each day, equal to the raw material inventory of the previous day plus raw materials purchased the previous day minus raw materials consumed the previous day;

IRₖ₁=IRAₖ,∀k=1, . . . ,K; (14);

where IRₖₜ is the inventory of raw material k at the beginning of the t^{th} day; and IRAₖ is the initial inventory of raw material k;

$${IR}_{kt} = {IR}_{k ( t - 1 )} + {RR}_{k ( t - 1 )} - {\sum }_{m = 1}^{M} ( {xg}_{km ( t - 1 )} \times {MG}_{km} \text{/} {β}_{k} ) , ∀ k = 1 , … , K , t = 1 , … , T ; ; \qquad ( 15 )$$

where RRₖₜ is the quantity of raw material k received at an end of the t^{th} day; MGₖₘ is the single-pot output of the granule k on granulator m; and βₖ is the capacity loss coefficient of production of granule k from raw materials.

Formula (16) represents that the raw materials required to produce granules per day plus the capacity loss cannot exceed the raw material inventory at the beginning of the day;

Σₘ₌₁^{M}(xgₖₘₜ×MGₖₘ/βₖ)≤IRₖₜ,∀k=1, . . . ,K,t=1, . . . ,T; (16).

Formulas (17)-(18) represent the granules inventory at the beginning of each day, which is equal to the granules inventory of previous day plus the granules produced on the previous day minus the granules consumed on the previous day.

IGₖ₁=IGAₖ,∀k=1, . . . ,K; (17);

where IGₖₜ is the inventory of granule k at the beginning of the t^{th} day;

IGₖₜ=IGₖ₍ₜ₋₁₎+Σₘ₌₁^{M}(xgₖₘ₍ₜ₋₁₎×MGₖₘ)−Σⱼ₌₁^{J}Σₙ₌₁^{N}(xt^{jn(t-1)}×MPⱼ×Bₖⱼ/γkj),∀k=1, . . . ,K,t=2, . . . ,T; (18);

where Bₖⱼ is the amount of granule k required to produce a unit of product j; γₖⱼ is the capacity loss coefficient of production of product j from granule k.

Formula (19) represents that the amount of granules required to produce the final product per day plus the capacity loss cannot exceed the granules inventory at the beginning of the day;

Σⱼ₌₁^{J}Σₙ₌₁^{N}(xtⱼₙₜ×MPⱼ×Bₖⱼ/γkj)≤IGₖₜ,∀k=1, . . . ,K,t=1, . . . ,T; (19).

Formula (20) represents that the number of molds used per day for tableting cannot exceed the total number of molds;

Σⱼ₌₁^{J}Σₙ₌₁^{N}(xtⱼₙₜ×MPⱼ×Bₖⱼ/γkj)≤IGₖₜ,∀k=1, . . . ,K,t=1, . . . ,T; (20);

where MODTⱼₛ represents a relationship between product and mold; when MODTⱼₛ is 1, it means that mold s is needed to produce product j; when MODTⱼₛ is 0, it means that mold s is not needed in production of product j; MODNₙₛ represents the relationship between machines and molds; when MODNₙₛ is 1, it means that tablet press n needs to the mold s; when MODNₙₛ is 0, it means that tablet press n does not need to mold s; MODₛ is the number of mold s; and s is the serial number of mold types, and is an integer selected from 1-S.

Formulas (21)-(22) represent the constraints on whether a product is produced and the production batches;

xtaⱼₙₜ≤xtⱼₙₜ,∀j=1, . . . ,J,n=1, . . . ,N,t=1, . . . ,T; (21);

M₁×xtaⱼₙₜ≥xtⱼₙₜ,∀j=1, . . . ,J,n=1, . . . ,N,t=1, . . . ,T; (22);

Formulas (23)-(24) represent the constraints on whether granules are produced and the production batches;

xgaₖₘₜ≤xgₖₘₜ,∀k=1, . . . ,K,m=1, . . . ,M,t=1, . . . ,T; (23);

where xgaₖₘₜ is 1 if granule k is produced on granulator m on the t^{th} day, otherwise xgaₖₘₜ is 0;

M₂×xgaₖₘₜ≥xgₖₘₜ,∀k=1, . . . ,K,m=1, . . . ,M,t=1, . . . ,T; (24).

Formulas (25)-(26) represent that the granulator and the tablet press cannot process more than a kind of granule and product on the same machine on the same day;

Σⱼ₌₁^{J}xtaⱼₙₜ≤1,∀n=1, . . . ,N,t=1, . . . ,T; (25);

Σₖ₌₁^{K}xgaₖₘₜ≤1,∀m=1, . . . ,M,t=1, . . . ,T; (26).

Formulas (27)-(28) represent whether the same mold is used for the product produced on the same tablet press on two adjacent days;

0≤savₙₜ≤1,∀n=1, . . . ,N,t=1, . . . ,T−1; (27);

savₙₜ≥Σⱼ₌₁^{J}(xtaⱼₙₜ−xtaⱼₙ₍ₜ₊₁₎)×MODTⱼₛ;∀n=1, . . . ,N,t=1, . . . ,T−1,s=1, . . . ,S; (28).

Formulas (29)-(31) represent whether the same product is produced on the same tablet press on two adjacent days;

0≤sav2ₙₜ≤1,∀n=1, . . . ,6,t=1, . . . ,T−1; (29);

where sav2ₙₜ is 1 if products produced on the same machine in two consecutive days are the same, otherwise sav2ₙₜ is 0;

sav2ₙₜ≥xtaⱼₙₜ−xtaⱼₙ₍ₜ₊₁₎,∀n=1, . . . ,6,t=1, . . . ,T−1,J=1, . . . ,J; (30);

sav2ₙₜ=1,∀n=7, . . . ,9,t=1, . . . ,T−1; (31).

Formula (32) represents the color change of the products produced on the same tablet press on two adjacent days;

Coprₙₜ≤Σⱼ₌₁^{J}(Corⱼ×xtaⱼₙ₍ₜ₊₁₎−Corⱼ×xtaⱼₙₜ/2)+1,∀n=1, . . . ,N,t=1, . . . ,T−1; (32);

where Corⱼ is the color attribute of product j, 1 means dark color, 0 means light color, and −1 means milky white.

Formulas (33)-(42) represent the conventional non-negative constraints, natural number constraints, and the 0-1 variable definition;

xtaⱼₙₜ∈{0,1},∀j=1, . . . ,J,n=1, . . . ,N,t=1, . . . ,T; (33);

xgaₖₘₜ∈{0,1},∀k=1, . . . ,K,m=1, . . . ,M,t=1, . . . ,T; (34);

vⱼₜ∈{0,1},∀j=1, . . . ,J,t=1, . . . ,T; (35);

wherein vⱼₜ is 1 if an order of the product j is delayed on the t^{th} day, otherwise vⱼₜ is 0;

Coprₙₜ∈{0,1},∀n=1, . . . ,N,t=1, . . . ,T−1; (36);

xgₖₘₜ∈N,∀k=1, . . . ,K,m=1, . . . ,M,t=1, . . . ,T; (37);

xtⱼₙₜ∈N,∀j=1, . . . ,J,n=1, . . . ,N,t=1, . . . ,T; (38);

IRₖₜ≥0,∀k=1, . . . ,K,t=1, . . . ,T; (39);

IGₖₜ≥0,∀k=1, . . . ,K,t=1, . . . ,T; (40);

sgₘₜ≥0,∀m=1, . . . ,M,t=1, . . . ,T; (41);

stₙₜ≥0,∀n=1, . . . ,N,t=1, . . . ,T; (42).

The objective function is constrained according to the constraint conditions to construct the production scheduling model.

S300 The order information and the production information are input into the production scheduling model to obtain a production scheduling scheme.

S400 The production scheduling model is verified through a simulation experiment.

The simulation experiment verification is performed on the developed production scheduling model to obtain the related experimental scheme, and the effectiveness of the scheduling model will be analyzed and verified through comparison between the experimental scheme and actual data, which are specifically described as follows.

Basic Data of Enterprise Operations

The relevant basic data will be collected based on the actual production situation of nutritional tablet, which mainly include the main equipment list of granules, the main equipment list of tablets, order information, the correspondence table of table nutrient and granules, tablet press corresponding to nutritional tablet, production speed, mold model, number of corresponding molds, production line and production efficiency corresponding to the granules, the validity period of the granules intermediate, granules inventory, manual scheduling data, etc.

Scheduling Model Simulation

With the minimum number of delayed batches as the objective, a mixed integer programming mathematical model is established based on the actual constraints and defined variables above. The scheduling model is solved using IBM ILOGCPLEXOptimization Studio V12.8.0 on a desktop computer with 3.60 GHz CPU and 16G memory, and then the numerical simulation is coded and run in MATLAB R2017a to obtain the scheduling scheme of the nutritional tablet (as shown in Table 1, the letters in the table indicate the code of the nutritional tablet). The scheme shows that the scheduling scheme output by the production scheduling model is consistent with the manual scheduling scheme, and the management personnel at production site can execute the scheduling scheme without additional training.

TABLE 1
Production scheduling sheet output by a production scheduling model

| | Nutrional | Nutrional | Nutrional | Nutrional | Nutrional | Nutrional | Nutrional |
|---|---|---|---|---|---|---|---|
| Date | tablet 1 | tablet 2 | tablet 4 | tablet 5 | tablet 8 | tablet 11 | tablet 12 |
| 2018 Jun. 1 | MT3K-4 | BT44-2 | MT69-3 | MTA4-3 | MTBV-2 | CT5K-3 | GT4R-2 |
| 2018 Jun. 2 | BT44-2 | GT3S-3 | GT3S-3 | MT3P-2 | MTA3-2 | BT43-3 | BT43-2 |
| 2018 Jun. 3 | GT3S-3 | BT4H-3 | GT3S-3 | MT69-3 | MTBW-2 | BT44-2 | MT3P-2 |
| 2018 Jun. 4 | MT67-3 | BT4J-3 | BT4J-3 | BT4J-3 | MT67-3 | MT67-3 | BT44-2 |
| 2018 Jun. 5 | MT3S-4 | MT3T-3 | BT44-2 | MT67-3 | BT4J-3 | BT4J-3 | BT3T-3 |
| 2018 Jun. 6 | MT71-2 | MT67-3 | BT4J-3 | BT44-2 | MT67-3 | MT3T-3 | MT67-3 |
| 2018 Jun. 7 | GT2L-2 | CTBC-2 | MT20-2 | MT3T-2 | GT3S-3 | BT44-2 | GT3S-3 |
| 2018 Jun. 8 | MT20-1 | CT3S-3 | BT44-2 | BT6D-1 | BT8M-3 | GT3S-3 | PT1D-1 |
| 2018 Jun. 9 | VT1B-1 | MT69-3 | BT8M-3 | BT44-2 | MT68-3 | GT3S-3 | GT3S-3 |

Comparative Analysis of Scheduling Scheme

In order to confirm the difference between the constructed scheduling model and the manual scheduling result, simulation experiment is performed on the scheme of the CPLEX model and the actual production data after sorting using MATLAB. Three main indicators are analyzed by comparing the delays, including the number of delays (cumulative 10,000 pieces), delayed batches (total of delayed batches) and average delay time. The comparison between manual production scheduling and model production scheduling is schematically illustrated in FIG. 4. It can be seen that the delay situation is obviously improved through the scheduling scheme obtained through the scheduling model. The goal of this embodiment is to minimize the delayed batches. According to the scheduling scheme obtained by the scheduling model, the improvement of delayed batches reached 52.7%.

The production scheduling method of this embodiment can significantly improve the delivery level of customer orders and improve the phenomenon of order delays without investing new labor or adding new equipment. Compared with manual scheduling, the scheduling method of this embodiment has the following beneficial effects.

1. Real-Time Operation

The production scheduling model is used to automatically schedule production. It only needs to analyze and clearly define the limiting factors of the factory when the production scheduling model is established, and then the computer can automatically consider all the definitions during the operation of the production scheduling model. The constraint condition can be updated iteratively at any time to achieve real-time scheduling.

2. Efficiency Improvement

In the scheduling model of this embodiment, it only takes a few hours to obtain the scheduling scheme at a time, while manual scheduling requires at least one day for experienced planners to iteratively update.

3. Collaborative Optimization

In the production process of nutritional tablet, the coordination of raw materials, testing, inventory, and granules production is required. Therefore, synergy effects must be considered in production scheduling. There are too many issues to be considered in manual scheduling and cannot be fully considered. Fortunately, the scheduling model can comprehensively consider many aspects, making multiple links linkage, mutual constraints and collaborative optimization.

4. Optimal Scheduling Scheme

The scheduling schemes are theoretically optimal under assumptions, minimizing the total batches of delayed delivery of nutritional tablet products orders, and greatly improving the production efficiency of nutritional tablet products. Manual scheduling is difficult to consider the overall situation, and can only ensure that the scheduling schemes are feasible, but cannot achieve optimality.

5. Reduction of the Operation Difficulty

The scheduling scheme output by the scheduling model has a simple format and easy to understand. Compared with manual scheduling, detailed scheduling is not need after a simple adjustment is made on the existing scheduling model. The scheduling result can be executed directly, which is a little different from industrial field operation.

6. Production scheduling can be automatically done using the production scheduling model. The tedious calculation work is executed by the computer, which not only has accurate and efficient calculation, but also has strong scalability. The constraint parameters can be adjusted and increased in real time, and rapid analysis and quantitative answer can be realized for the problems or updates encountered in the process of production and operation.

7. In addition to the production scheduling applied to orders, the production scheduling model can also be used to calculate various factory operation optimization plans, which can provide sufficient theoretical data support, clarify the optimization direction and priority and help to analyze the input and output of each optimization scheme.

This embodiment solves the scheduling problem of nutritional tablet products, and at the same time has important reference values for improving the production efficiency of other products, the level of order delivery and customer satisfaction. The improvement of the delivery level can bring positive impacts to various aspects, such as refined management of raw material suppliers, improvement of inventory turnover efficiency, improvement of customer satisfaction, reduction of product costs, etc., which can improve the competitiveness of the whole supply chain.

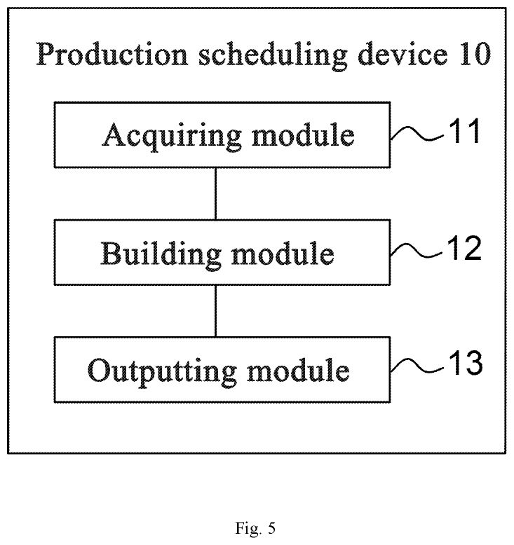

Referring to an embodiment shown in FIG. 5, a production scheduling device 10 specifically includes an acquiring module 11, a building module 12 and an outputting module 13.

The acquiring module 11 is configured to obtain pending order information and production information of the nutritional tablets, and constraint conditions jointly formed by the pending order information and the production information.

The building module 12 is configured to build a production scheduling model according to the constraint conditions.

The outputting module 13 is configured to input the pending order information and the production information into the production scheduling model to output a production scheduling scheme.

In an embodiment, the production scheduling device 10 further includes a verification module, configured to verify the production scheduling model through a simulation experiment.

It should be noted that the specific implementation process of the production scheduling device in this embodiment is the same as that of the production scheduling method. For details, please refer to the embodiment of the production scheduling method, which will not be repeated here.

The above are only the preferred embodiments of the present disclosure, and are not intended to limit the scope of the present disclosure. Any changes, modifications and improvements made by those skilled in the art without departing from the spirit of the present disclosure shall fall within the scope of the present disclosure defined by the appended claims.

What is claimed is:

1. A method for production scheduling of nutritional tablets, comprising:
acquiring pending order information and production information of the nutritional tablets, and constraint conditions jointly formed by the pending order information and the production information;
constructing a production scheduling model according to the constraint conditions; and
inputting the pending order information and the production information into the production scheduling model to obtain a production scheduling scheme.

2. The method of claim 1, wherein the construction of the production scheduling model according to the constraint condition is performed through steps of:
constructing an objective function as shown in formula (1):
min Σⱼ₌₁^{J}Σₜ₌₁^{T}(pⱼ×vⱼₜ); (1);
wherein j is a serial number of products, and is an integer selected from 1-J; t is a time number with a unit of day, and production is arranged from a first day; t is an integer selected from 1-T; T is an order production scheduling cycle; pⱼ is a production priority of product j; and Uⱼₜ is a delay amount of product j on the t^{th} day; and
the constraint conditions comprising formulas (2)-(42) are shown as follows:
s.t. Σₖ₌₁^{K}(xgₖₘₜ×PGTₖₘ+xgaₖₘₜ×SATGₘ)+sgₘₜ=24,∀m=1, . . . ,M,t=1, . . . ,T,WDₜ=1; (2);
wherein s is a serial number of mold types, and s is an integer selected from 1-S; k is a serial number of granules, and k is an integer selected from 1-K; xgₖₘₜ is the number of batches of granules k processed on a granulator m on the t^{th} day; PGTₖₘ is a one-pot processing time of the granules k; a processing time of granules that cannot be processed by granulator m is regarded as 0; SATGₘ is time for single clearing on the granulator m during switching of granules; sgₘₜ is an idle time of granulator m at the t^{th} day; m is a serial number of a granulator, and m is an integer selected from 1-M; and WDₜ is a date for rest;
Σ_{j=1,MP}_{ⱼ}_{≤150}^{J}(xtⱼₙₜ×(MPⱼ/PTⱼₙ+SITₙ/SCₙ)+xtaⱼₙₜ×SATTₙ−xtaⱼₙₜ×SITₙ/SCₙ)+Σ_{j=1,MP}_{ⱼ}_{>150}^{J}(xtⱼₙₜ×(MPⱼ/PTⱼₙ+SITₙ)+xtaⱼₙₜ×SATTₙ−xtaⱼₙₜ×SITₙ)+stₙₜ=24+Coprₙₜ×CLTₙ+(1−savₙₜ)×SAVTTNₙ+(1−sav2ₙₜ)×SAVTTNₙ;n=1, . . . 5,t=1 . . . ,T−1,WDₜ=1 (3);
wherein MPⱼ is a single-batch output of the product j; for two machines manufacturing the same product in a workshop, output of the two machines is calculated as double an one-pot output of one of the two machines; xtⱼₙₜ is the number of batches of the product j manufactured on a tablet press n on the t^{th} day; PTⱼₙ is a processing speed of the product j on the tablet press n; a processing speed of a product that fails to be processed by the tablet press n is regarded as 0; SITₙ is time for a single small cleaning for the tablet press n; SCₙ is a frequency of small cleaning for the tablet press n, which is calculated by the number of batches of the same product that have been produced when one small cleaning is required; xtaⱼₙₜ is 1 if the product j is produced on the tablet press n on the t^{th} day, otherwise xtaⱼₙₜ is 0; SATTₙ is the longest time used for a single large clearing during product switching on the tablet press n; stₙₜ is an idle time of the tablet press n on the t^{th} day; Coprₙₜ is 1 if a color of a product produced on the same machine on two consecutive days is changed from dark to light, otherwise Coprₙₜ is 0; CLTₙ is a time required for switching color of a product on the tablet press n; savₙₜ is 1 if the same mold is used on the same machine for two consecutive days, otherwise savₙₜ is 0; SAVTTₙ is a time required for mold switching on the tablet press n, and is also the time for large clearing; and n is a serial number of the tablet press, and is an integer selected from 1-N;
$$\begin{matrix}{\sum }_{j = 1 , {MP}_{j} \leq 150}^{J} ( {xt}_{jnt} \times ( {MP}_{j} \text{/} {PT}_{jn} + {SIT}_{n} \text{/} {SC}_{n} ) + {xta}_{jnt} \times {SATT}_{n} - {xta}_{jnt} \times {SIT}_{n} \text{/} {SC}_{n} ) + {\sum }_{j = 1 , {MP}_{j} > 150}^{J} ( {t}_{jnt} \times ( {MP}_{j} \text{/} {PT}_{jn} + {SIT}_{n} ) + {xta}_{jnt} \times {SATT}_{n} - {xta}_{jnt} \times {SIT}_{n} ) + {st}_{nt} = 24 n = 1 , … , 5 , t = T , {WD}_{t} = 1 ; ; & ( 4 ) \\ {\sum }_{j = 1 , {MP}_{j} \leq 150}^{J} ( {xt}_{jnt} \times ( {MP}_{j} \text{/} {PT}_{jn} + {SIT}_{n} \text{/} {SC}_{n} ) + {xta}_{jnt} \times {SATT}_{n} - {xta}_{jnt} \times {SIT}_{n} \text{/} {SC}_{n} ) + {\sum }_{j = 1 , {MP}_{j} > 150}^{J} ( {t}_{jnt} \times ( {MP}_{j} \text{/} {PT}_{jn} + {SIT}_{n} ) + {xta}_{jnt} \times {SATT}_{n} - {xta}_{jnt} \times {SIT}_{n} ) + {st}_{nt} = 24 + {Copr}_{nt} \times {CLT}_{n} + ( 1 - {sav}_{nt} ) \times {SAVTTN}_{n} + ( 1 - sav {2}_{nt} ) \times {SAVTTN}_{n} ; n = 6 , … , 9 , t = 1 , … , T - 1 , {WD}_{t} = 1 ; ; & ( 5 ) \\ {\sum }_{j = 1 , {MP}_{j} \leq 150}^{J} ( {xt}_{jnt} \times ( {MP}_{j} \text{/} {PT}_{jn} + {SIT}_{n} \text{/} {SC}_{n} ) + {xta}_{jnt} \times {SATT}_{n} - {xta}_{jnt} \times {SIT}_{n} \text{/} {SC}_{n} ) + {\sum }_{j = 1 , {MP}_{j} > 150}^{J} ( {t}_{jnt} \times ( {MP}_{j} \text{/} {PT}_{jn} + {SIT}_{n} ) + {xta}_{jnt} \times {SATT}_{n} - {xta}_{jnt} \times {SIT}_{n} ) + {st}_{nt} = 24 n = 6 , … , 9 , t = T , {WD}_{t} = 1 ; ; & ( 6 ) \\ {\sum }_{j = 1}^{J} {\sum }_{n = 1}^{N} {xta}_{jnt} = 0 , ∀ t = 1 , … , T , {WD}_{t} = 0 ; ; & ( 7 ) \\ {\sum }_{t = 1}^{{FX}_{j} - 1} {\sum }_{n = 1}^{N} {xta}_{jnt} = 0 , ∀ j = 1 , … , J ; ; & ( 8 )\end{matrix}$$
wherein FXⱼ is a release date of raw materials, and j is an integer selected from 1-J;
IFⱼ₁=0,∀j=1, . . . ,J; (9);
wherein IFjt is an inventory of the product j at a beginning of the t^{th} day;
IFⱼₜ=IFⱼ₍ₜ₋₁₎+Σₙ₌₁^{N}(xtⱼₙ₍ₜ₋₁₎×MPⱼ)−DMTⱼ₍ₜ₋₁₎,∀j=1, . . . ,J,t=2, . . . ,T+1; (10);
wherein DMTⱼₜ is a demand of the product j at the t^{th} day;
uⱼₜ≥−IFⱼ₍ₜ₊₁₎,∀j=1, . . . ,J,t=1, . . . ,T; (11);
uⱼₜ≥0,∀j=1, . . . ,I,t=1, . . . ,T; (12);
uⱼₜ/MPⱼ≤vⱼₜ×M₃,∀j=1, . . . ,J,t=1, . . . ,T; (13);
wherein M₁₋₃ is a constant;
IRₖ₁=IRAₖ,∀k=1, . . . ,K; (14);
wherein IRₖₜ is an inventory of raw material k at the beginning of the t^{th} day; and IRAₖ is an initial inventory of the raw material k;
IRₖₜ=IRₖ₍ₜ₋₁₎+RRₖ₍ₜ₋₁₎−Σₘ₌₁^{M}(xgₖₘ₍ₜ₋₁₎×MGₖₘ/βₖ),∀k=1, . . . ,K,t=1, . . . ,T; (15);
wherein RRₖₜ is a quantity of the raw material k received at an end of the t^{th} day; MGₖₘ is a single-pot output of the granule k on the granulator m; and βₖ is a capacity loss coefficient of production of granule k from raw materials;
Σₘ₌₁^{M}(xgₖₘₜ×MGₖₘ/βₖ)≤IRₖₜ,∀k=1, . . . ,K,t=1, . . . ,T; (16);
IGₖ₁=IGAₖ,∀k=1, . . . ,K; (17);
wherein IGₖₜ is an inventory of the granule k at the beginning of the t^{th} day;
IGₖₜ=IGₖ₍ₜ₋₁₎+Σₘ₌₁^{M}(xgₖₘ₍ₜ₋₁₎×MGₖₘ)−Σⱼ₌₁^{J}Σₙ₌₁^{N}(xt^{jn(t-1)}×MPⱼ×Bₖⱼ/γkj),∀k=1, . . . ,K,t=2, . . . ,T; (18);
wherein Bₖⱼ is an amount of the granule k required to produce a unit of the product j; γₖⱼ is a capacity loss coefficient of production of the product j from the granule k;
Σⱼ₌₁^{J}Σₙ₌₁^{N}(xtⱼₙₜ×MPⱼ×Bₖⱼ/γkj)≤IGₖₜ,∀k=1, . . . ,K,t=1, . . . ,T; (19);
Σⱼ₌₁^{J}Σₙ₌₁^{N}(xtⱼₙₜ×MPⱼ×Bₖⱼ/γkj)≤IGₖₜ,∀k=1, . . . ,K,t=1, . . . ,T; (20);
wherein MODTⱼₛ represents a relationship between products and molds; when MODTⱼₛ is 1, it means that a mold s is needed to produce the product j; when MODTⱼₛ is 0, it means that the mold s is not needed in production of the product j; MODNₙₛ represents a relationship between machine and mold; when MODNₙₛ is 1, it means that the tablet press n needs to use the mold s; when MODNₙₛ is 0, it means that the tablet press n does not need to use the mold s; MODₛ is the number of the mold s; and s is a serial number of mold types, and is an integer selected from 1-S;
xtaⱼₙₜ≤xtⱼₙₜ,∀j=1, . . . ,J,n=1, . . . ,N,t=1, . . . ,T; (21);
M₁×xtaⱼₙₜ≥xtⱼₙₜ,∀j=1, . . . ,J,n=1, . . . ,N,t=1, . . . ,T; (22);
xgaₖₘₜ≤xgₖₘₜ,∀k=1, . . . ,K,m=1, . . . ,M,t=1, . . . ,T; (23);
wherein xgaₖₘₜ is 1 if the granule k is produced on the granulator m on the t^{th} day, otherwise xgaₖₘₜ is 0;
M₂×xgaₖₘₜ≥xgₖₘₜ,∀k=1, . . . ,K,m=1, . . . ,M,t=1, . . . ,T; (24);
Σⱼ₌₁^{J}xtaⱼₙₜ≤1,∀n=1, . . . ,N,t=1, . . . ,T; (25);
Σₖ₌₁^{K}xgaₖₘₜ≤1,∀m=1, . . . ,M,t=1, . . . ,T; (26);
0≤savₙₜ≤1,∀n=1, . . . ,N,t=1, . . . ,T−1; (27);
savₙₜ≥Σⱼ₌₁^{J}(xtaⱼₙₜ−xtaⱼₙ₍ₜ₊₁₎)×MODTⱼₛ;∀n=1, . . . ,N,t=1, . . . ,T−1,s=1, . . . ,S; (28);
0≤sav2ₙₜ≤1,∀n=1, . . . ,6,t=1, . . . ,T−1; (29);
wherein sav2ₙₜ is 1 if products produced on the same machine in two consecutive days are the same, otherwise sav2ₙₜ is 0;
sav2ₙₜ≥xtaⱼₙₜ−xtaⱼₙ₍ₜ₊₁₎,∀n=1, . . . ,6,t=1, . . . ,T−1,J=1, . . . ,J; (30);
sav2ₙₜ=1,∀n=7, . . . ,9,t=1, . . . ,T−1; (31);
Coprₙₜ≤Σⱼ₌₁^{J}(Corⱼ×xtaⱼₙ₍ₜ₊₁₎−Corⱼ×xtaⱼₙₜ/2)+1,∀n=1, . . . ,N,t=1, . . . ,T−1; (32);
wherein Corⱼ is a color attribute of the product j, 1 means dark color, 0 means light color, and −1 means milky white;
xtaⱼₙₜ∈{0,1},∀j=1, . . . ,J,n=1, . . . ,N,t=1, . . . ,T; (33);
xgaₖₘₜ∈{0,1},∀k=1, . . . ,K,m=1, . . . ,M,t=1, . . . ,T; (34);
vⱼₜ∈{0,1},∀j=1, . . . ,J,t=1, . . . ,T; (35);
wherein vⱼₜ is 1 if an order of the product j is delayed on the t^{th} day, otherwise vⱼₜ is 0;
Coprₙₜ∈{0,1},∀n=1, . . . ,N,t=1, . . . ,T−1; (36);
xgₖₘₜ∈N,∀k=1, . . . ,K,m=1, . . . ,M,t=1, . . . ,T; (37);
xtⱼₙₜ∈N,∀j=1, . . . ,J,n=1, . . . ,N,t=1, . . . ,T; (38);
IRₖₜ≥0,∀k=1, . . . ,K,t=1, . . . ,T; (39);
IGₖₜ≥0,∀k=1, . . . ,K,t=1, . . . ,T; (40);
sgₘₜ≥0,∀m=1, . . . ,M,t=1, . . . ,T; (41); and
stₙₜ≥0,∀n=1, . . . ,N,t=1, . . . ,T; (42); and
constraining the objection function according to the constraint conditions to construct the production scheduling model.

3. The method of claim 1, wherein the pending order information comprises an order time, a product name, a product quantity, an order priority and a delivery date.

4. The method of claim 1, wherein the production information comprises a machine list information, a machine scheduling information, a mold information, a processing speed information of each machine, a cleaning rule information, a customer priority information, a workshop personnel scheduling information, a product information, an auxiliary equipment information, a follow-up inspection and logistics time information, information of an output target and a scheduling cycle, a product batch information, and a raw material inventory and arrival information.

5. The method of claim 1, wherein the production scheduling model is verified by a simulation experiment.

6. A device for production scheduling of nutritional tablets, comprising:
an acquiring module;
a building module; and
an outputting module;
wherein the acquiring module is configured to obtain pending order information, production information of the nutritional tablets, and constraint conditions jointly formed by the pending order information and the production information;
the building module is configured to build a production scheduling model according to the constraint conditions; and
the outputting module is configured to input the pending order information and the production information into the production scheduling model to output a production scheduling scheme.

7. The device of claim 6, further comprising:
a verification module, configured to verify the production scheduling model through a simulation experiment.

8. An electronic device, comprising:
a processor; and
a memory;
wherein the memory stores a program code; and the processor is configured to execute the method of claim 1 by executing the program code.

9. A computer-readable storage medium, comprising:
a program code;
wherein the program code is configured to drive an electronic device to execute the method of claim 1 when being run on the electronic device.
